(19) United States
(12) Patent Application Publication
(10) Pub. No.: US 2021/0015535 A1
(43) Pub. Date: Jan. 21, 2021

(54) Cold Plasma Therapy Device with Replaceable Dielectric Barrier

(71) Applicants: Qun Li, Newark, DE (US); Sean Xiaolu Wang, Wilmington, DE (US)

(72) Inventors: Qun Li, Newark, DE (US); Sean Xiaolu Wang, Wilmington, DE (US)

(73) Assignee: BWT Property, Inc., Newark, DE (US)

(21) Appl. No.: 16/905,991

(22) Filed: Jun. 19, 2020

Related U.S. Application Data
(60) Provisional application No. 62/874,228, filed on Jul. 15, 2019.

(51) Int. Cl. A61B 18/00

(52) U.S. Cl. CPC A61B 18/00; A61B 2018/00583; A61B 2018/00886; A61B 2018/00892; A61B 2018/00648

(57) ABSTRACT

This invention discloses a DBD plasma therapy device with replaceable dielectric barrier for treating different patients with different medical conditions. The plasma therapy device is equipped with a variety of dielectric barriers. The dielectric barriers may have different electrical characteristics (which are determined by their materials as well as physical dimensions and shapes) to adapt for the treatment of different types of biological tissues. The dielectric barrier of the plasma therapy device can be replaced to avoid contamination and cross-infection. As an additional feature, the plasma device further comprises an optical sensor, such as a spectroscopic sensor, for monitoring the emission spectrum of the plasma discharge. The emission spectrum can be utilized to analyze the composition of the reactive species generated by the plasma discharge and provide feedback control to the plasma therapy device.

### REFERENCE TO RELATED APPLICATION

This application claims the inventions which were disclosed in Provisional Patent Application No. 62/874,228, filed Jul. 15, 2019, entitled “COLD PLASMA THERAPY DEVICE WITH REPLACEABLE DIELECTRIC BARRIER”. The benefit under 35 USC § 119(e) of the above mentioned United States Provisional Applications is hereby claimed, and the aforementioned application are hereby incorporated herein by reference.

### FIELD OF THE INVENTION

This invention generally relates to a plasma therapy device, and more specifically, to a cold plasma therapy device with a replaceable dielectric barrier.

### BACKGROUND

Plasma as the fourth fundamental state of matter, is a neutral ionized gas composed of positively charged ions, electrons, and neutral particles. In common thermal plasma, all particles approach thermal equilibrium due to intensive collisions between electrons and heavy particles. The temperature in such plasma can reach several thousand degrees. On the other hand, there is another type of plasma in which electrons and heavy particles are in thermal non-equilibrium. In this case, the temperature of the heavy particles is much lower than that of the electrons. This type of plasma is called non-thermal plasma or cold plasma. The heavy particle temperature in cold plasma is typically between 25° C. and 45° C. The plasma discharge may take place in ambient air or in specially supplied gas flow. Many reactive species, including oxygen-based radicals, nitrogen-based radicals, and other components, are generated in the cold plasma. This complicated chemistry can lead to a variety of interactions between cold plasma and biological tissues, allowing the cold plasma to be used for biomedicine.

Dielectric barrier discharge (DBD), which involves electrical discharge between two electrodes separated by an insulating dielectric barrier, is one effective method to produce cold plasma. For biomedical applications, the living tissue is often employed as one of the electrodes, and the plasma discharge is produced between the dielectric barrier and the subject tissue. When the DBD device is used for treating different patients, it is highly desirable to replace the dielectric barrier between treatments to avoid cross-infection. Also, it is desirable to switch among different types of dielectric barriers for treating different medical conditions. This is because the effective treatment of one specific medical condition may require a specific combination of reactive species in the plasma discharge which in turn is affected by both the electrical characteristics of the subject tissue and the parameters of the dielectric barrier. Currently, there is no DBD plasma therapy device providing easily replaceable or switchable dielectric barriers.

### SUMMARY OF THE INVENTION

It is the overall goal of the present invention to solve the above-mentioned problems and provide a DBD plasma therapy device with replaceable dielectric barrier for treating different patients with different medical conditions. The plasma therapy device is equipped with a variety of dielectric barriers. The dielectric barriers may have different electrical characteristics (which are determined by their materials as well as physical dimensions and shapes) to adapt for the treatment of different types of biological tissues. The dielectric barrier of the plasma therapy device can be replaced to avoid contamination and cross-infection. As an additional feature, the plasma device further comprises an optical sensor, such as a spectroscopic sensor, for monitoring the emission spectrum of the plasma discharge. The emission spectrum can be utilized to analyze the composition of the reactive species generated by the plasma discharge and provide feedback control to the plasma therapy device.

### BRIEF DESCRIPTION OF THE FIGURES

The accompanying figures where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the DBD plasma therapy device with a replaceable dielectric barrier; and

FIG. 2 illustrates another exemplary embodiment of the DBD plasma therapy device.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a cold plasma therapy device. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

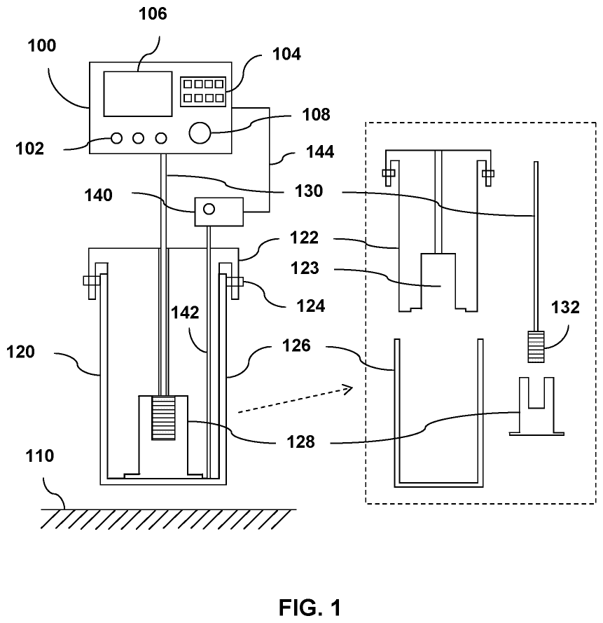

FIG. 1 illustrates one exemplary embodiment of the DBD cold plasma therapy device. The plasma therapy device comprises a high voltage power supply 100, which supplies high voltage to a DBD probe 120 (not drawn to scale) through a high voltage cable 130. The power supply 100 is preferably a pulsed power supply with adjustable output voltage, repetition rate, and duty cycle. The pulse width of the power supply is preferably in the nanosecond to millisecond range. The output voltage is preferably in the kilovolt to hundreds of kilovolt range. The power supply 100 comprises adjustment knobs 102 and keypads 104 for the user to control the output voltage, repetition rate, and duty cycle as well as a display 106 to display the current value of these parameters. The power supply 100 further comprises an emergency switch 108 for shutting down the unit in case of an emergency. For example, a sensor circuitry may be employed to detect the misplacement or crack of the dielectric barrier and shut down the unit if these happened. The DBD probe 120 comprises four major components: a first dielectric barrier 122, a second dielectric barrier 126, an electrode 128, and a high voltage cable 130. An exploded view of these components is shown on the right of FIG. 1. The first dielectric barrier 122 has a cavity 123 to hold the electrode 128 in place. The electrode 128 is preferably made of a highly conductive material, such as copper or aluminum. The second dielectric barrier 126, which is replaceable and switchable on the treatment site, can be mounted onto the first dielectric barrier 122 and the electrode 128 and secured by a plurality of set screws 124 or other fastening means so as to enclose the first dielectric barrier 122 and the electrode 128, hence insulating the electrode 128 from the subject biological tissue 110. High voltage is supplied from the power supply 100 to the electrode 128 through the high voltage cable 130, the end of which is soldered to a metal screw 132 and affixed into the top of the electrode 128. In this exemplary embodiment, the thickness of the bottom wall of the second dielectric barrier 126 is selected such that when the subject biological tissue 110 is positioned within a fixed distance from the bottom wall, a plasma discharge of predetermined intensity will be produced under the supplied voltage. The thickness of the sidewall of the first dielectric barrier 122 and the second dielectric barrier 126 is selected such that no plasma discharge is produced even when the subject biological tissue is in contact with the sidewall of the second dielectric barrier 126. The first dielectric barrier 122 is preferably made of plastic material, while the second dielectric barrier 126 can be made of plastic, glass or other dielectric materials depending on application requirements.

Due to the diversity of the medical conditions and types of biological tissues (e.g., different body parts of the human or animal subject) to be treated as well as the variations from individual to individual, a customized treatment protocol with specific plasma density, reactive species composition, and dosage may be required to achieve the optimum therapeutic outcome. These plasma parameters are determined by the output voltage, repetition rate, duty cycle, and treatment time of the power supply 100, the composition of the gas in which the plasma discharge takes place, the distance between the second dielectric barrier 126 and the subject biological tissue 110, and also affected by the electrical characteristics (e.g., capacitance, resistance, inductance) of the subject biological tissue 110 and the second dielectric barrier 126, and the grounding condition of the subject biological tissue 110. The electrical characteristics of the biological tissue are further determined by its composition, volume, and humidity. The electrical characteristics of the second dielectric barrier 126 are mainly determined by its material (hence dielectric constant or relative permittivity) as well as its physical dimensions and shapes (especially thickness). The switchable second dielectric barrier 126 offers additional freedom for controlling the properties of the plasma discharge as its capacitance affects the discharge voltage, and its dielectric constant affects the streamer intensity, diameter, and density of the plasma discharge. The replaceable second dielectric barrier 126 also prevents contamination and/or cross-infection from one patient to another patient. A set of replaceable and switchable second dielectric barriers 126, each having different or similar electrical characteristics, can be provided to fulfill the above purposes of (i) controlling the properties of the plasma discharge, and (ii) preventing contamination and/or cross-infection. For practical applications, it is desirable to establish a correlation between the medical conditions and biological tissues to be treated and the corresponding parameters of the high voltage power supply 100 and the switchable second dielectric barrier 126, the gas flow composition, the distance between the DBD probe 120 and the subject tissue 110, etc. The correlation can be in the form of a look-up table, which is stored in the memory of the plasma therapy device. Before plasma treatment, the operator selects the optimum treatment protocol from the look-up table based on the conditions of the subject biological tissue. The high voltage power supply 100 and the DBD probe 120 are then adjusted to provide cold plasma therapy at the optimum treatment protocol.

To further ensure the therapeutic outcome, the plasma therapy device is equipped with an optical spectroscopic sensor 140 for monitoring the emission spectrum of the plasma discharge. Referring to FIG. 1, the optical emission from the plasma discharge is collected by one or more optical fibers 142 (which are embedded inside the first dielectric barrier 122) and delivered into the spectroscopic sensor 140. The spectroscopic sensor 140 obtains a spectrum of the optical emission and determines the composition and concentration of the reactive species in the cold plasma based on the spectrum. This information is used to provide feedback control 144 to the power supply 100 such that its output voltage, repetition rate, duty cycle, and treatment time is automatically adjusted to obtain the optimum therapeutic effect. In a slight variation of the present embodiment, the electrode 128 may have a meshed structure such that the optical fiber 142 can be placed on top of (or inside) the electrode 128 to collect the optical emission of the cold plasma. In addition, an imaging sensor, in combination with an imaging fiber, may be used for monitoring images of the plasma discharge to provide the feedback control information. Free space optics may be used instead of optical fibers for optical signal collection for both the spectroscopic sensor and the imaging sensor.

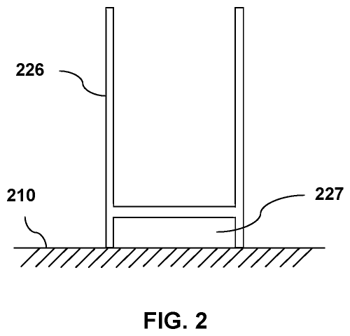

In another exemplary embodiment of the DBD plasma therapy device as shown in FIG. 2, the replaceable second dielectric barrier 226 of the DBD probe has an additional cavity 227 formed by its bottom and sidewalls. The cavity 227 forms an enclosure when the DBD probe is placed in contact with the subject biological tissue 210 and covers the area to be treated. When a high voltage is applied to the electrode of the BDB probe, plasma discharge takes place inside the enclosure. In comparison with the open-air environment, this enclosed environment favors the production of certain reactive species, such as nitrogen oxides (NOₓ), which are beneficial for the treatment of certain medical conditions. In a slight variation of the present embodiment, a layer of hydrogel (alginate, gelatin, etc.) is applied to the bottom surface of the second dielectric barrier 226 (the hydrogel may either fill up the cavity 227 or not). The hydrogel is enriched with oxygen and nitrogen to facilitate the generation of reactive oxygen and nitrogen species (RONS) under the plasma discharge. One advantage of this approach is that the produced RONS can be maintained in the hydrogel for a long period of time to provide continued treatment to the subject tissue even after the plasma discharge is off.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and figures are to be regarded in an illustrative rather than a restrictive sense, and all such modifications are intended to be included within the scope of the present invention. The benefits, advantages, and solutions to problems, and any element(s) that may cause any benefit, advantage, or solution to occur or become more pronounced are not to be construed as a critical, required, or essential features or elements of any or all the claims. The invention is defined solely by the appended claims, including any amendments made during the pendency of this application and all equivalents of those claims as issued.

What is claimed is:

1. A dielectric barrier discharge (DBD) cold plasma therapy device for treating a subject, the plasma therapy device comprising:
a high voltage power supply for supplying a high voltage to an electrode; and
a set of replaceable and switchable dielectric barriers, each being mountable onto the electrode, wherein one of the set of replaceable and switchable dielectric barriers is mounted onto to the electrode to insulate the electrode from the subject;
wherein a cold plasma discharge is produced between the mounted dielectric barrier and the subject for treating the subject when the high voltage is supplied to the electrode.

2. The plasma therapy device of claim 1, wherein the high voltage power supply is a pulsed, high voltage power supply with adjustable output voltage, repetition rate, and duty cycle.

3. The plasma therapy device of claim 1, wherein the electrode is enclosed by the mounted dielectric barrier.

4. The plasma therapy device of claim 1, wherein the set of replaceable and switchable dielectric barriers have different physical dimensions.

5. The plasma therapy device of claim 1, wherein the set of replaceable and switchable dielectric barriers have different physical shapes.

6. The plasma therapy device of claim 1, wherein the set of replaceable and switchable dielectric barriers are made of different materials.

7. The plasma therapy device of claim 1, further comprising an optical spectroscopic sensor for measuring the optical emission of the cold plasma discharge to obtain an emission spectrum and determining a property of the cold plasma discharge based on the emission spectrum.

8. The plasma therapy device of claim 7, wherein the optical spectroscopic sensor provides feedback control to the high voltage power supply based on the determined property of the cold plasma discharge.

9. The plasma therapy device of claim 1, wherein at least one of the set of replaceable and switchable dielectric barriers, has a cavity to form an enclosure covering an area of the subject when being placed in contact with the subject.

10. The plasma therapy device of claim 9, wherein a layer of hydrogel is applied to the surface of the cavity of the at least one dielectric barrier.
